(19) United States
(12) Patent Application Publication
(10) Pub. No.: US 2022/0064624 A1
(43) Pub. Date: Mar. 3, 2022

(54) HYDROGEL-ENCAPSULATED CELLS AND HYDROGEL-DISPERSED CELLS

(71) Applicant: UNIVERSITY OF WYOMING, Laramie, WY (US)

(72) Inventors: Benjamin NOREN, Laramie, WY (US); John OAKEY, Laramie, WY (US); Zhongliang JIANG, Laramie, WY (US); Kun JIANG, Laramie, WY (US)

(21) Appl. No.: 17/463,362

(22) Filed: Aug. 31, 2021

Related U.S. Application Data
(60) Provisional application No. 63/073,005, filed on Sep. 1, 2020.

(51) Int. Cl. C12N 11/089; C12N 11/087

(52) U.S. Cl. CPC C12N 11/089; C12N 11/087

(57) ABSTRACT

Embodiments of the present disclosure generally relate to compositions that include hydrogel-encapsulated/dispersed cells, compositions including hydrogel-encapsulated/dispersed cells, and to processes for forming such hydrogel-encapsulated/dispersed cells and compositions thereof. The compositions can be used for, e.g., therapeutic applications. In some examples, the hydrogel-encapsulated/dispersed cells are formed using photoreactive groups chemically attached to polyethylene glycol to form a material which, upon exposure to a desired wavelength or wavelength range of light, reacts to form a cross-linked hydrogel network.

### CROSS-REFERENCE TO RELATED APPLICATIONS

This application claims priority to U.S. Provisional Patent Application 63/073,005, filed Sep. 1, 2020, which is herein incorporated by reference in its entirety.

### GOVERNMENT RIGHTS

This invention was made with government support under the Faculty Early Career Development Program (BBBE 1254608) awarded by the National Science Foundation. The government has certain rights in the invention.

### BACKGROUND

### Field

Embodiments of the present disclosure generally relate to compositions that include hydrogel-encapsulated/dispersed cells, compositions including hydrogel-encapsulated/dispersed cells, and to processes for forming such hydrogel-encapsulated/dispersed cells and compositions thereof. The compositions can be used for, e.g., therapeutic applications.

### Description of the Related Art

Cellular therapies including stem cells, platelet-rich plasma, and bone marrow aspirate have been investigated as candidates to regenerate damaged cartilage, epithelial, connective, and nervous tissues. Such therapies act by stimulating endogenous progenitor cells to regenerate a target tissue through secretion of trophic factors. Though an area of intense interest due to its promise of improving and reversing a wide variety of conditions including those that are currently untreatable, these strategies have limited efficacy in patients due to, e.g., poor viability of injected cells and short retention times at the desired therapeutic site. Various strategies to improve the regenerative capacity of therapeutic cells have been proposed and researched, but such strategies typically require invasive surgical procedures or induce undesirable cell behavior.

One less invasive strategy involves the use of delivering living cells encapsulated in a polymer. However, conventional methods for encapsulation drastically reduce cell viability and efficacy. This problem has precluded long-term encapsulation applications with suitable materials and at necessary length scales for sufficient nutrient and trophic factor diffusion. It has also prevented development of a minimally invasive method of delivering polymerized cell-laden hydrogels to an injury site.

There is a need for improved compositions, and processes for making such compositions, that overcome one or more deficiencies in the art.

### SUMMARY

Embodiments of the present disclosure generally relate to compositions that include hydrogel-encapsulated/dispersed cells, compositions including hydrogel-encapsulated/dispersed cells, and to processes for forming such hydrogel-encapsulated/dispersed cells and compositions thereof. The compositions can be used for, e.g., therapeutic applications.

In an embodiment, a composition is provided. The composition includes a microcapsule comprising a core and a polymeric shell enclosing the core, the core comprising a cell, and the polymeric shell comprising, in polymerized form, one or more photoreactive monomers and a linker.

In another embodiment, a composition that includes hydrogel-encapsulated/dispersed cells is provided. The composition includes a hydrogel comprising, in polymerized form, one or more photoreactive monomers and a thiol linker, wherein at least one of the one or more photoreactive monomers comprise a methylene functional group. The composition further includes cells dispersed within the hydrogel.

In another embodiment, a process for forming hydrogel-encapsulated/dispersed cells is provided. The process includes introducing cells with one or more components in a microfluidic device to form a reaction mixture. The process further includes polymerizing the reaction mixture by exposure to ultraviolet light, under polymerization conditions, to form a composition comprising the cells dispersed in or encapsulated within a hydrogel.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a schematic of an example device for forming a hydrogel-encapsulated/dispersed cell according to at least one embodiment of the present disclosure.

FIG. 2A is an exemplary image of the polymerized hydrogels encapsulating a cell according to at least one embodiment of the present disclosure.

FIG. 2B is an exemplary image showing example hydrogel-encapsulated/dispersed cells according to at least one embodiment of the present disclosure.

FIG. 3 is a flowchart showing selected operations of an example process for forming hydrogel-encapsulated/dispersed cells according to at least one embodiment of the present disclosure.

FIG. 4 is an exemplary image showing fluorescent cells in the hydrogels according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments of the present disclosure generally relate to compositions that include hydrogel-encapsulated/dispersed cells, compositions including hydrogel-encapsulated/dispersed cells, and to processes for forming such hydrogel-encapsulated/dispersed cells and compositions thereof. The compositions can be used for, e.g., therapeutic applications. Briefly, the compositions include a hydrogel formed from the polymerization of photoreactive monomers. A cell or a plurality of cells can be encapsulated, dispersed, suspended, retained, or otherwise held in the hydrogels. The inventors have found that these compositions can, e.g., enhance survival of the cells, improve retention of the biomolecules cells, control delivery of the cells, and control gene expression of therapeutic cells. Moreover, the compositions described herein can also be injected in a minimally invasive way.

In some examples, processes described herein generally include introducing one or more cells, one or more polymerizable monomers, and an oil (e.g., a fluorocarbon oil) to a microfluidic device. Due to physical interactions between oil and the other components introduced to the microfluidic device, droplets having the cells and polymerizable species therein are formed. The droplets travel containing the cells and polymerizable monomers are then exposed to ultraviolet (UV) light as they travel through the microfluidic device. The UV light polymerizes the one or more polymerizable monomers into a cross-linked hydrogel network encapsulating/dispersing the cells in, e.g., microscopic hydrogel spheres or sphere-like hydrogels. If desired, hydrogels containing the cells can be isolated and re-suspended for use in, e.g., therapeutic applications including injection and topical administration.

As discussed above, conventional cellular therapies for regenerating, e.g., damaged tissues, have limited efficacy because of poor viability of the injected therapeutic cells and short retention times at the desired therapeutic site. Further, conventional strategies to improve the viability of the therapeutic cells can induce undesirable cell behavior when implanted in the patient. Moreover, the dosage form administered to the patient can require invasive surgical procedures placing the patient at undue risk.

In contrast, and as described herein, the hydrogels can provide a unique environment that mimics the characteristics of a cell's natural endogenous extracellular matrix and cell-microenvironment effects. This enables the encapsulated/dispersed cells to, e.g., maintain a high level of viability on a long-term basis comparable to standard monoculture, prevent cell migration, and/or permit control of cellular cytokine expression. Such improvements can contribute to faster and more complete tissue healing than the current state-of-the-art. As such, the compositions enabled herein can have greater efficacy than conventional cellular therapies. For therapeutic uses, embodiments described herein enable, e.g., controlled release of the cell or exosome from the hydrogel network for targeted therapies for, e.g., humans and animals. Moreover, embodiments described herein facilitate high-throughput generation of hydrogel particles (such as microparticles and nanoparticles).

In addition, a previously unsolved problem applying encapsulation and dispersion technology to living cells is the drastic reduction in cell viability after polymerization in these microenvironments. This problem has precluded long-term encapsulation and dispersion applications with suitable materials and at necessary length scales for sufficient nutrient and trophic factor diffusion. It has also prevented development of a minimally invasive method of delivering polymerized cell-laden hydrogels to an injury site. Here, it is believed that the production of cytotoxic radical oxygen species (ROS) is a main factor in reducing the cell's viability and efficacy. Moreover, the presence of cytotoxic ROS at micron-length scales that are oftentimes advantageous when encapsulating cells, is considerably greater than at bulk scales due to rapid oxygen diffusion during the polymerization reaction to encapsulate or disperse cells using conventional technologies.

In some embodiments, such problems are overcome by using certain photoreactive monomer(s), such as polyethylene glycol norbornene (PEGNB), to form a PEG-based hydrogel. As a non-limiting illustration, photoreactive monomer(s) can have alkene (or methylene) groups can react with thiol monomer(s) by a step-growth polymerization reaction between an “ene” portion of the photoreactive monomer(s) and a thiol of the thiol monomer(s). The inventors show that such a step-growth polymerization mitigates and actively eliminates the ROS that would otherwise drastically reduce cell viability when encapsulating them.

Further, when using, e.g., PEGNB monomers, the resulting polymer can be characterized as a more homogenous hydrogel network with reduced network contraction than other equivalent materials in the art, and thus further reduces stress upon encapsulated and/or dispersed cells. Adjusting, e.g., the quantity, molecular mass, and ratio of the thiol and ene components, as well as photocatalyst concentration and UV light exposure intensity and time, can enable control over the resultant hydrogel cross-linking properties and its hydrolytic degradation over time.

While the present disclosure refers to “microcapsules”, “microgels”, and “microparticles”, it will be appreciated that the disclosure may be applied to capsules, gels, and particles having a smaller size (e.g., “nanocapsules”, “nanogels”, or “nanoparticles”) or capsules, gels, and having a larger size (e.g., “macrocapsules”, “macrogels”, or “macroparticles”). In addition, while embodiments and examples are described herein in terms of cells, it is contemplated that other objects, including biomolecules, can be utilized. For example, the processes described herein can be used to form hydrogel-encapsulated “exosome” biomolecules and used to form therapeutic doses of hydrogel-encapsulated exosome biomolecules.

Also, while embodiments and examples are described herein with reference to hydrogel encapsulation of cells, it is contemplated that the cells can additionally, or alternatively, be suspended, dispersed, retained, or otherwise held in the hydrogels. For example, device 100 described below can be utilized to form hydrogels having cells dispersed therein, and processes for forming the hydrogel-encapsulated cells can be used to form hydrogels having cells dispersed therein.

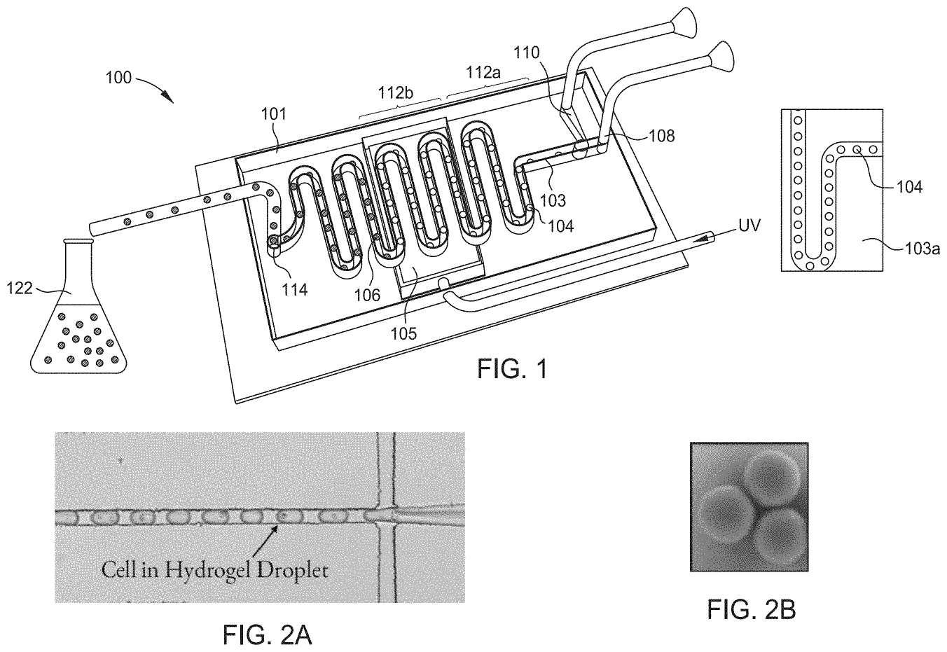

FIG. 1 is a schematic of an example device 100 for forming hydrogel-encapsulated/dispersed cells according to at least one embodiment of the present disclosure. Such hydrogel-encapsulated/dispersed cells produced can be in the form of microparticles. Device 100 can be used for continuous production of hydrogel-encapsulated/dispersed cells.

Device 100 includes a microfluidic device 101 having a fluidic channel 103. In at least one embodiment, the fluidic channel 103 has a diameter of micrometers (μm) to millimeters (mm). For example, the fluidic channel 103 has a diameter from about 1 μm to about 2 mm and/or a depth of about 1 μm to about 2 mm. One or more portions of the fluidic channel 103 can be in the form of loops, discussed below. The fluidic channel 103 includes a mixing area 112a where a hydrogel forming solution, discussed below, can be mixed with cells and oil, and a polymerization area 112b where monomers of the hydrogel forming solution polymerize to form hydrogels that encapsulate and/or disperse the cells.

As stated above, portions of the fluidic channel 103 can be in the form of loops. The loops enable control over, e.g., the kinetics of mixing, the kinetics of polymerization, the exposure time for polymerization, and/or the gelation of the hydrogels. That is, the loops can enable uniform processing of microparticles. Other morphologies or shapes besides, or in addition to, loops are contemplated to enable processing of the microparticles. Such morphologies or shapes include spirals or other tortuous paths. That is, any suitable morphology or shape that extends the length of the fluidic channel 103 in, e.g., the mixing area 112a and/or the polymerization area 112b would have the same or similar effect of controlling the exposure time so that the desired cross-linking can be achieved on a microfluidic chip with high-throughput droplet production capabilities.

The microfluidic device 101 has an opening 110 for introducing a hydrogel forming solution to the fluidic channel 103. The hydrogel forming solution includes photoinitiators, reaction components, and/or photoreactive monomers (e.g., PEG-dithiol linker, PEGNB, PEGDA, PLA, etc.). Cells in, e.g., a buffer, can be introduced to the fluidic channel 103 via opening 110 or a separate opening. The microfluidic device 101 includes another opening 108 for introducing a suspension fluid to the fluidic channel 103. The suspension fluid can be an oil, such as a fluorocarbon oil. The oil can serve to pinch off the cells and hydrogel forming solution (e.g., photoinitiators, reaction components, and/or photoreactive monomers) into droplets and carry the droplets through the microfluidic device 101. Openings 108 and 110 are coupled to the fluidic channel 103. As shown, tubings are coupled to the individual openings 108, 110 to allow introduction of the oil, cells, hydrogel forming solution, and/or other reaction components to the fluidic channel 103 of the microfluidic device 101. However, it is contemplated that introduction of the oil, cells, hydrogel forming solution, and/or other reaction components to the microfluidic device 101 can be performed in other suitable ways, such as direct connecting Leuer lock type devices, snap-together microfluidic assemblies, and syringe-like devices, without departing from the scope of the present disclosure.

Although two openings are described, more or less openings can be used to introduce the oil, cells, hydrogel forming solution, and/or other reaction components to the microfluidic device 101. The inset identified as 103a is a pictorial representation of the fluidic channel 103 showing droplets 104 in suspension fluid (e.g., the oil). The droplets 104 can include, but are not limited to, cells, photoreactive monomers, photoinitiators, reaction components, and/or fluorocarbon oil, as well as other materials.

The fluidic channel 103 includes the polymerization area 112b. At the polymerization area 112b, monomers and/or reaction components of the droplets 104 polymerize to form, e.g., a hydrogel 106, that suspends, disperses, encapsulates, retains, or otherwise holds a cell or a plurality of cells. As shown, the fluidic channel 103 of the polymerization area 112b includes a suitable number of loops (and/or other suitable shape) to enable, e.g., sufficient polymerization of the monomers and other reaction components as well as sufficient gelation of the hydrogels.

The device 100 further includes a polymerization control device 105 optically and/or mechanically coupled to at least a portion of the fluidic channel 103. The polymerization control device 105 is configured to cause a polymerization reaction when the desired materials are within the polymerization area 112b. The polymerization control device 105 can include a UV-light source(s), such as a UV lamp, UV light source concentrated via lenses and/or microscope objective, or laser, that polymerizes the monomers and/or reaction components to form the hydrogel (e.g., hydrogels 106). Coupling of the polymerization control device 105 can take multiple forms. For example, the microfluidic device 101 can be placed on top of, below, or otherwise adjacent to, the polymerization control device 105. The UV light source can be located in a stand-alone unit outside of the microfluidic device 101.

FIG. 2A is an exemplary image of the polymerized hydrogels within the fluidic channel 103 of the polymerization area 112b. A portion of the image shows a cell in a hydrogel droplet. After polymerization, the cell-laden microparticles (e.g., the hydrogel-encapsulated/dispersed cells) move toward the fluidic channel exit 114 where the cell-laden microparticles can be collected via any suitable collection unit 122, e.g., flask, centrifuge tube, reservoir, vessel, or the like. FIG. 2B is an exemplary image showing the hydrogel-encapsulated/dispersed cells collected from the microfluidic device 101. Other materials (byproducts, suspension fluid, unreacted materials, etc.) can exit the fluidic channel exit 114 along with the hydrogel-encapsulated/dispersed cells. Accordingly, the hydrogel-encapsulated/dispersed cells or compositions comprising the hydrogel encapsulated/dispersed cells can be purified, or otherwise isolated, from the other materials exiting the microfluidic device 101.

Movement of the various materials (e.g., suspension fluid, and cells, photoreactive monomers, photoinitiators, and/or reaction components, etc.) from the one or more openings 108, 110 to the fluidic channel exit 114 can be controlled by, e.g., capillary action, laminar flow, temperature, a pumping mechanism (e.g., a syringe pump, pressure pump, or piezoelectric pump), electrodes, and the like. Such elements controlling the movement can be placed at either opposing ends of the device, opposite ends, or along various regions along a length of the fluidic channel 103.

In some embodiments, the hydrogel-encapsulated/dispersed cells formed by embodiments described herein can be in the form of a microcapsule. This microcapsule can include a core and a polymeric shell which at least partially encloses the core. The core includes a cell or a plurality of cells. The polymeric shell of the microcapsule is formed by the polymerization of one or more photoreactive monomers and one or more linkers as described below.

Cells suspended, dispersed, encapsulated, retained, or otherwise held in the hydrogel particles include, but are not limited to, mesenchymal stem cells (MSCs), mesenchymal stromal cells, perinatal cells, fat derived stem cells, bone marrow aspirate concentrate, chondrocytes, regulatory T cells, and beta cells. It is contemplated that any other suitable cell can also be suspended in the hydrogel particles.

As discussed above, the photoreactive monomers used to form the hydrogel contain photoreactive functional groups chemically attached to, e.g., polyethylene glycol (PEG). Illustrative, but non-limiting, examples of photoreactive functional groups include alkenes, thiols acids, or combinations thereof. Upon irradiation, the photoreactive monomers (with or without co-reactants, such as linkers described below) polymerize to form a hydrogel.

Non-limiting examples of photoreactive monomers include, but are not limited to, polyethylene glycol norbornene (PEGNB), polyethylene glycol diacrylate (PEGDA), derivatives thereof, or combinations thereof. The photoreactive monomers can be branched (e.g., ˜20 k 4-arm PEGNB and ˜40 k 8-arm PEGNB) or unbranched. Other PEG-based derivatives having varied reactive functional groups are also contemplated. The molecular weight and shape (e.g., number of arms on PEGNB) of one or more photoreactive monomers, among other characteristics, can be changed.

Photoreactive monomers can also include non-PEG-based monomers such as acrylates, acids (e.g., lactic acid, hyaluronic acid), gelatin, collagen, or combinations thereof. For example, polylactic acid (PLA) and derivatives thereof can be used. Block copolymers and triblock copolymers can also be used such as triblock PLA and PLA-PEG-PLA.

Molecular conformation of the photoreactive monomers can be varied to impart desired material properties to the hydrogel microenvironment. For example, 4-arm or 8-arm molecular structures such as 4-arm PEGNB and 8-arm PEGNB can be utilized. Further, the chemical properties of the hydrogel microenvironment can be modified via click chemistry through addition of thiolated agents (for, e.g., PEGNB) or similar acrylated agents (for, e.g., PEGDA) such as thiolated or acrylated cell adhesion peptides like RGD (arginine-glycine-aspartate) or CRGDS (cystine-arginine-glycine-aspartate-serine). Mixtures of one or more photoreactive monomers, e.g., a mixture of PEGNB and PEGDA) can also be used, as well as mixtures that include non-PEG-based photolabile hydrogels such as gelatin methacrylate and/or photolabile hyaluronic acid.

A molecular weight of the one or more photoreactive monomers can be from about 250 Da to about 50,000 Da, such as from about 5,000 Da to about 50,000 Da, such as from about 10,000 Da to about 45,000 Da, such as from about 15,000 Da to about 40,000 Da, such as from about 20,000 Da to about 35,000 Da, such as from about 25,000 Da to about 30,000 Da. Illustrative, but non-limiting, examples of the molecular weight of the photoreactive monomer are from about 250 Da to about 10,000 Da, such as from about 500 Da to about 9,000 Da, such as from about 1,000 Da to about 8,000 Da, such as from about 2,000 Da to about 7,000 Da, such as from about 3,000 Da to about 6,000 Da, such as from about 4,000 Da to about 5,000 Da. In some examples, the molecular weight of the one or more photoreactive monomers is 30,000 Da or less. Higher or lower molecular weights of the one or more photoreactive monomers are contemplated. The molecular weight of the photoreactive monomer refers to the number average molecular weight (Mₙ). The Mₙ is the Mₙ provided by the manufacturer of the photoreactive monomer. The photoreactive monomers can be introduced to the microfluidic device 101 in the form of a hydrogel forming solution. The hydrogel forming solution can contain one or more photoreactive monomers, one or more photoinitiators, one or more linkers, one or more cell adhesion peptides, or combinations thereof, as well as additional components. Suitable organic and/or aqueous solvents are utilized as a portion of the hydrogel forming solution. Such organic and/or aqueous solvents can include water, saline, phosphate buffered saline, appropriate biologically compatible liquid, or combinations thereof.

The concentration of the one or more photoreactive monomers useful for the hydrogel forming solution can be from about 5 wt % to about 75 wt %, such as from about 10 wt % to about 70 wt %, such as from about 15 wt % to about 65 wt %, such as from about 20 wt % to about 60 wt %, such as from about 25 wt % to about 55 wt %, such as from about 30 wt % to about 50 wt %, such as from about 35 wt % to about 45 wt %. In at least one embodiment, the concentration of the one or more photoreactive monomers in the hydrogel forming solution is from about 5 wt % to about 35 wt %, such as from about 10 wt % to about 30 wt %, such as from about 15 wt % to about 25 wt %, based on a total weight percent of the hydrogel forming solution (not to exceed 100 wt %). Higher or lower concentrations of the one or more photoreactive monomers can be used depending on application.

The components that are subjected to polymerization can further include a one or more linkers, such as a dithiol linkers, such as a polyethylene glycol-dithiol (PEG-dithiol), a derivative thereof, or combinations thereof. PEG-dithiol is a thiolated PEG having two thiol groups. The linker can be referred to as a thiol-containing monomer or dithiol linker unless the context indicates otherwise. When a dithiol linker is utilized, the photoreactive monomer(s) polymerize with the thiol-containing monomer(s) via a step-growth polymerization reaction occurring between the ene portion of the monomers and the thiol of the thiol-containing monomer.

A molecular weight of the one or more linkers can be from about 500 Da to about 10,000 Da, such as from about 1,000 Da to about 9,500 Da, such as from about 1,500 Da to about 9,000 Da, such as from about 2,000 Da to about 8,500 Da, such as from about 2,500 Da to about 8,000 Da, such as from about 3,000 Da to about 7,500 Da, such as from about 3,500 Da to about 7,000 Da, such as from about 4,000 Da to about 6,500 Da, such as from about 4,500 Da to about 6,000 Da, such as from about 5,000 Da to about 5,500 Da. In some examples, the molecular weight of the linker is about 6,000 Da or less, such as from about 500 Da to about 6,000 Da, such as from about 1,000 Da to about 5,000 Da, such as from about 1,500 Da to about 4,500 Da, such as from about 2,000 Da to about 4,000 Da, such as from about 2,500 Da to about 3,500 Da. The molecular weight of the linker refers to the number average molecular weight (Mₙ). The Mₙ is the Mₙ provided by the manufacturer of the linker.

Higher or lower molecular weights of the one or more linkers are contemplated. Illustrative, but non-limiting, examples of PEG-dithiol linkers include ˜1.5 k PEG-dithiol, 3.5 k PEG-dithiol, and ˜5 k PEG-dithiol.

A concentration of the one or more linkers (e.g., PEG-dithiol) in the hydrogel forming solution can be from about 1 mM to about 50 mM, such as from about 5 mM to about 45 mM, such as from about 10 mM to about 40 mM, such as from about 15 mM to about 35 mM, such as from about 20 mM to about 30 mM, based on a total molar concentration of the hydrogel forming solution. Higher or lower concentrations of the one or more linkers can be used depending on application.

The hydrogel forming solution can also include one or more photoinitiators. Illustrative, but non-limiting, examples of photoinitiators include lithium phenyl-2,4,6-trimethylbenzoylphosphinate (LAP) photoinitiator, 2-hydroxy-2-methyl propiophenone (e.g., Irgacure™ 1173, Darocur™ 1173), and combinations thereof. A concentration of the one or more photoinitiators in the hydrogel forming solution can be from about 0.0001 wt % to about 1 wt %, such as from about 0.001 wt % to about 0.9 wt %, such as from about 0.01 wt % to about 0.5 wt %, such as from about 0.05 wt % to about 0.1 wt %, based on the total wt % of the hydrogel forming solution. Higher or lower concentrations of the one or more photoinitiators can be used depending on, e.g., the application or desired results.

The hydrogel forming solution can also include one or more cell adhesion peptides such as RGD, CRGDS, or a combination thereof. A concentration of the one or more cell adhesion peptides in the hydrogel forming solution can be from about 0.5 mM to about 10 mM, such as from about 1 mM to about 8 mM, such as from about 2 mM to about 6 mM, such as from about 3 mM to about 4 mM.

Cells in a suitable media such as an aqueous buffer dulbecco's modified eagles media (DMEM), such as phosphate buffered saline, are also introduced to the microfluidic device 101. A concentration of cells in the suitable media introduced or otherwise delivered to the microfluidic device 101 can be from about 1 cell/mL to about 1×10⁹ cells/mL, such as from about 1×10³ cells/mL to about 1×10⁸ cells/mL, such as from about 1×10⁵ cells/mL to about 1×10⁷ cells/mL. A higher or lower concentration of cells in the suitable media can be utilized.

Additional reaction components such as reaction mixture precursors, solvents, catalysts, reagents, and the like, can be introduced to the microfluidic device 101. These additional reaction can mix and/or interact (e.g., chemically and/or physically) with the components of the hydrogel forming solution, the cells, and/or the oil to form the hydrogel-encapsulated cells.

Using the components described above, various formulations can be used to form the hydrogel-encapsulated/dispersed cells and compositions thereof. The formulation can be that of the hydrogel forming solution or separate solutions that are introduced to the microfluidic device.

A non-limiting formulation useful for the polymerization can include (a) from about 0.1 wt % to about 40 wt %, such as from about 1 wt % to about 40 wt %, such as from about 5 wt % to about 35 wt %, such as from about 10 wt % to about 20 wt % of one or more photoreactive monomers, such as a PEGNB, ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (b) from about 1 mM to about 100 mM, such as from about 5 mM to about 50 mM PEG dithiol ranging in molecular weight from about 100 Da to about 10,000 Da; and/or (c) from about 0.0001 wt % to about 1 wt %, such as from about 0.01 wt % to about 0.1 wt % of LAP photoinitiator. Additional components can be used as desired.

When PEGNB is utilized with a second photoreactive monomer such as PEGDA, PLA, PLA-PEG-PLA, etc., a non-limiting formulation can include the aforementioned formulation with about 0.1 wt % to about 40 wt %, such as from about 1 wt % to about 40 wt %, such as from about 5 wt % to about 35 wt %, such as from about 10 wt % to about 20 wt % of the second photoreactive monomer (e.g., PEGDA, PLA, PLA-PEG-PLA, etc.) having a molecular weight from about 1,000 Da to about 30,000 Da, such as from about 5,000 Da to about 20,000 Da, such as from about 10,000 Da to about 15,000 Da. Additional components can be used as desired.

An illustrative, but non-limiting, formulation useful to form a PEGPLA/NB composite hydrogels can include: (a) from about 0.1 wt % to about 40 wt %, such as from about 1 wt % to about 40 wt %, such as from about 5 wt % to about 35 wt % such as from about 10 wt % to about 20 wt % of a first photoreactive monomer (e.g., PLA-PEG-PLA, etc.) having a molecular weight from about 1,000 Da to about 30,000 Da, such as from about 5,000 Da to about 20,000 Da, such as from about 10,000 Da to about 15,000 Da; (b) from about 0.1 wt % to about 40 wt %, such as from about 1 wt % to about 40 wt %, such as from about 5 wt % to about 35 wt %, such as from about 10 wt % to about 20 wt % of a second photoreactive monomer (e.g., PEGNB, such as 4-arm PEGNB, 8-arm PEGNB, or a combination thereof) ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (c) from about 1 mM to about 100 mM, such as from about 5 mM to about 50 mM PEG dithiol ranging in molecular weight from about 100 Da to about 10,000 Da; and/or (d) from about 0.0001 wt % to about 1 wt %, such as from about 0.01 wt % to about 0.1 wt % of the LAP photoinitiator.

Embodiments of the present disclosure also generally relate to processes for forming hydrogel-encapsulated/dispersed cells or compositions comprising hydrogel-encapsulated/dispersed cells. Briefly, and in some examples, the process generally includes forming a reaction mixture that includes a cell and one or more photoreactive monomers, and then polymerizing the reaction mixture to form the hydrogel-encapsulated/dispersed cells or compositions comprising hydrogel-encapsulated/dispersed cells. In some embodiments, processes of forming hydrogel-encapsulated/dispersed cells include forming droplets, having cells and polymerizable species therein, within an oil in a microfluidic device.

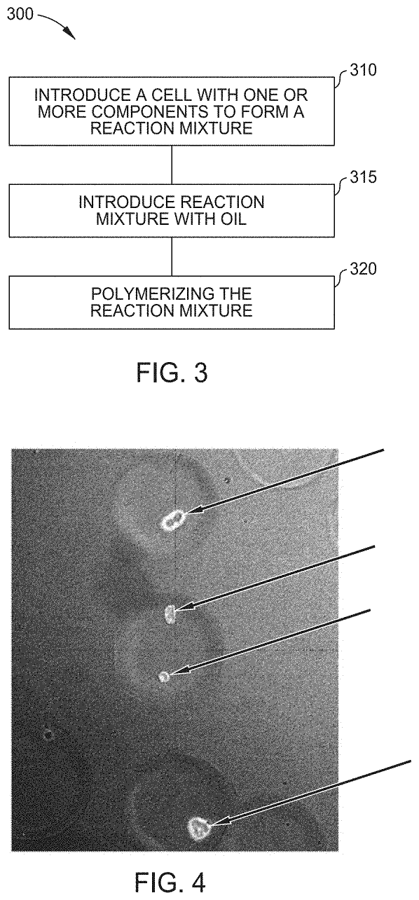

FIG. 3 is a flowchart showing selected operations of an example process 300 for forming hydrogel-encapsulated/dispersed cells or compositions thereof. Process 300 can be performed in a microfluidic device such as the microfluidic device 101. Process 300 begins at operation 310 with introducing a cell with one or more components in the microfluidic device 101 to form a reaction mixture. The cell can be in the form of a suspension in an aqueous buffer such as phosphate buffered saline (PBS). The one or more components can include one or more photoreactive monomers, one or more linkers (e.g., dithiol linker), one or more photoinitiators, and/or one or more solvents. Other materials such as reagents, catalysts, and/or cell-adhesion peptides can be optionally added. The reaction mixture can be in the form of microparticles in solution in the presence of the oil. These microparticles can be created in sizes ranging from, e.g., about 1 μm to about 2000 μm, such as from about 2 μm to about 1000 μm, such as from about 4 μm to about 500 μm, with cell concentrations ranging from, e.g., 1 cell per microparticle to thousands of cells per microparticle, or more.

Operation 310 can include flowing a hydrogel forming solution into the microfluidic device 101 at a rate of about 0.1 μL/min to about 150 μL/min, such as from about 25 μL/min to about 125 μL/min, such as from about 50 μL/min to about 100 μL/min, such as from about 80 μL/min to about 100 μL/min. Higher or lower rates are contemplated for the hydrogel forming solution. Operation 310 can further include flowing a cell stream—a cell in a suspension such as a buffer, such as PBS—into the microfluidic device 101 at a rate of about 0.1 μL/min to about 150 μL/min, such as from about 25 μL/min to about 125 μL/min, such as from about 50 μL/min to about 100 μL/min, such as from about 80 μL/min to about 100 μL/min. Higher or lower rates are contemplated for this cell stream. In some embodiments, the hydrogel forming solution and the cell stream are introduced at the same time or separate times to the same or different opening of the microfluidic device.

At operation 315, an oil (e.g., a fluorocarbon oil) can be introduced to the reaction mixture. Upon introduction, the oil with the reaction mixture can form droplets. Here, for example, the oil is added to the microfluidic device 101, and the oil can aid in the formation of droplets within the fluidic channel. Such droplets help, e.g., bring together the polymerizable reactants and the cells. A flow rate of the oil into the microfluidic device 101 can be from about 0.1 μL/min to about 200 μL/min, such as from about 1 μL/min to about 150 μL/min, such as from about 25 μL/min to about 125 μL/min, such as from about 50 μL/min to about 100 μL/min, such as from about 80 μL/min to about 100 μL/min. Higher or lower rates are contemplated for this oil stream.

Adjusting the initial cell titer as well as channel dimensions and flowrates as described herein can enable control of microparticle size and cell concentration in an independent manner.

The process 300 further includes polymerizing the reaction mixture to form a hydrogel-encapsulated/dispersed cells, or compositions thereof, at operation 320. The polymerization reaction of operation 320 can be performed under polymerization conditions. Polymerization of the reaction mixture forms the hydrogel-encapsulated/dispersed cells and/or compositions comprising hydrogel-encapsulated/dispersed cells. In some embodiments, the pH of the reaction mixture before, during, and/or after polymerization can be from about 5 to about 9, such as from about 6 to about 8, such as from about 6.5 to about 7.5.

Polymerization conditions can include exposing the reaction mixture to UV light at a desired wavelength or wavelength range, such as a wavelength or wavelength range of about 320 nm to about 460 nm, such as from about 340 nm to about 440 nm, such as from about 360 nm to about 420 nm, such as from about 380 nm to about 400 nm or from about 400 nm to about 420 nm, for varying timespans. The wavelength or wavelength range can be constant or varying during operation 320. The source of the UV light can be the polymerization control device 105 described above. It is contemplated that other wavelengths of light can be used with appropriate reacting photoinitiators.

The polymerization conditions of operation 320 can further include a duration of exposure to the UV light. Such durations can be 1 millisecond (ms) or more and/or about 5 min. or less, such as from about 1 ms to about 60 seconds (s), such as from about 5 milliseconds to about 50 seconds, such as from about 50 milliseconds to about 45 seconds, such as from about 100 milliseconds to about 40 seconds, such as from about 0.5 seconds to about 30 seconds, such as from about 1 second to about 20 seconds. Shorter or longer durations of exposure to UV light are contemplated.

An energy density of the UV light for the polymerization conditions of operation 320 can be from about 1 mW/cm² to about 10,000 mW/cm², such as from about 10 mW/cm² to about 1,000 mW/cm², such as from about 50 mW/cm² to about 500 mW/cm², such as from about 75 mW/cm² to about 150 mW/cm², such as from about 80 mW/cm² to about 120 mW/cm². Higher or lower energy densities are contemplated. The energy density can be constant or varying during operation 320.

The droplets can enable the polymerized cells to be injected in a minimally invasive manner (e.g., through a syringe) analogous to “naked” cells. This removes the need for surgical procedures and greatly reduces the chance of complications and the patient recovery time. Also the droplets can maintain superior oxygenation of encapsulated/dispersed cells and enable superior waste removal from the immediate cell environment, as opposed to a “bulk” hydrogel containing cells. This is due to the superior surface are to volume ratio which facilitates rapid diffusion between the encapsulated cell and the surrounding.

The polymerization process described herein improves cell viability over conventional techniques. For example, upon photoinitiation, a homogenous hydrogel network with reduced network contraction relative to other equivalent materials reduces stress imparted on encapsulated/dispersed cells. In addition, it is believed that the polymerization described herein can mitigate ROS through active participation in the cross-linking mechanism of, e.g., PEGNB, contributing to the polymerization of the network rather than removing electrons from cellular membranes and destabilizing them, which is what kills or contributes to cell death. In polymerizations with PEGDA, ROS can be mitigated by purging oxygen from the microenvironment via a non-reactive or inert gas which is free or substantially free of oxygen can be used, such as nitrogen and noble gases (e.g., argon). For polymerizations using mixtures of PEGDA and PEGNB, ROS can be mitigated by the addition of PEGNB and its above properties, but can be further mitigated if necessary through purging of the microenvironment with inert gas.

In some cases, the combination of PEGNB with another photoreactive monomer, such as PEGDA, enables physical and chemical tuning of the droplet environment to optimize cell viability and excretion of, e.g., cytokines. The encapsulation/dispersion process and resultant hydrogel can maintain cell viability longer than non-encapsulated/dispersed counterparts, and can localize cells at a target location by temporarily preventing their migration.

After polymerization, the hydrogel-encapsulated/dispersed cells or compositions comprising the hydrogel-encapsulated/dispersed cells can be purified or otherwise isolated from the other materials exiting the microfluidic device.

The hydrogels formed herein can have an average diameter of about 1 μm to about 2000 μm, such as from about 2 μm to about 1000 μm, such as from about 4 μm to about 500 μm, as determined by ImageJ (National Institutes of Health). In at least one embodiment, the hydrogel can have an average diameter of about 500 μm or less, such as from about 50 μm to about 450 μm, such as from about 100 μm to about 400 μm, such as from about 150 μm to about 350 μm, such as from about 200 μm to about 300 μm. In some embodiments, the hydrogel can have an average diameter of about 50 μm to about 200 μm, such as from about 100 μm to about 180 μm or from about 75 μm to about 125 μm.

Cell concentrations within the hydrogel (e.g., suspended, dispersed, encapsulated, retained, or otherwise held in the hydrogels) can range from about 1 cell per hydrogel to thousands of cells per hydrogel, or more.

The following examples are put forth so as to provide those of ordinary skill in the art with a complete disclosure and description of how to make and use aspects of the present disclosure, and are not intended to limit the scope of aspects of the present disclosure. Efforts have been made to ensure accuracy with respect to numbers used (e.g. amounts, dimensions, etc.) but some experimental errors and deviations should be accounted for.

### Examples

Example 1a: The hydrogel forming solution included about 20 wt % 4-arm 20 k PEGNB, about 20 mM 3.5 k PEG dithiol, about 0.6 wt % LAP. Cells were then mixed with the hydrogel forming solution on-chip in the fluidic channel of the microfluidic device 101. Cell-laden 10 wt % PEGNB microgels were fabricated under constant flow rate of 0.5 μL/min) while varying oil phase flow rate to about 2 μL/min, about 5 μL/min, and about 20 μL/min.

Example 1b: A hydrogel forming solution includes ˜7 wt % 8-arm 40 k PEGNB with ˜3 mM 5 k PEG-dithiol, and ˜3 mM RGD and/or CRGDS. Cells were then mixed with the hydrogel forming solution on-chip in the fluidic channel of the microfluidic device 101. Flow rates utilized are shown in Example 1a.

Example 2: The hydrogel forming solution included about 20 wt % 4-arm 20 k PEGNB, about 20 mM 3.5 k PEG dithiol, and about 0.6 wt % LAP. Cells were then mixed with the hydrogel forming solution on-chip in the fluidic channel of the microfluidic device 101. Cell-laden 10 wt % PEGNB microgels were fabricated under a constant flow rate of 0.5 μL/min while varying oil phase flow rate to about 2 μL/min, about 5 μL/min, and about 20 μL/min.

Example 3: PEGNB is mixed with dithiol linker, LAP, and cell-containing culture media to a final concentration of ˜10 wt % PEGNB, ˜10 mM dithiol linker, and ˜0.1 wt % LAP for preparing ˜10 wt % PEGNB hydrogels. To vary macromer concentrations, ˜20 wt % PEGNB, ˜20 mM dithiol linker, ˜0.1 wt % LAP were mixed for preparing ˜20 wt % PEGNB hydrogels, and ˜30 wt % PEGNB, ˜30 mM dithiol linker, ˜0.1 wt % LAP were mixed for preparing ˜30 wt % PEGNB hydrogels. Cells were then mixed with the hydrogel forming solution on-chip in the fluidic channel of the microfluidic device 101. Flow rates utilized are shown in Example 1a.

Example 4: PEGDA hydrogel forming solution was mixed to a final concentration of ˜10 wt % PEGDA (Mₙ 3400 Da, JenKem Technology) and ˜0.1 wt % LAP. To vary PEGDA concentrations, ˜20 wt % and ˜30 wt % PEGDA were mixed with ˜0.1 wt % LAP for polymerization. Cells were then mixed with the hydrogel forming solution on-chip in the fluidic channel of the microfluidic device 101. Flow rates utilized are shown in Example 1a.

Examples 1a, 1b, 2, 3, and 4 all formed hydrogel-encapsulated/dispersed cells that showed good cell viability.

FIG. 4 shows fluorescent cells (indicated by the arrows) in the hydrogels, illustrating that the processes described herein form hydrogel-encapsulated/dispersed cells.

The processes described herein utilize precise control of, e.g., the cross-linking density, pore size, and mechanical properties of the microparticles to tune diffusive properties of the microenvironment, enabling optimal exchange of nutrients and trophic factors between the encapsulated cell(s) and their bulk surroundings. The processes described herein enable the creation of cell-laden microparticles that maintain high viability—analogous to that of unencapsulated control—regardless of microparticle size. The processes also enable the encapsulated cells to maintain this high level of viability on a long-term basis, comparable to standard monoculture. The microparticle environment offers a cross-linked hydrogel mesh network that mimics the characteristics of a cell's natural endogenous extracellular matrix and cell-microenvironment effects.

The hydrogels or compositions comprising hydrogels described herein have a biocompatible microenvironments suitable for encapsulation and/or dispersion of living cells in sufficient quantities and are formed in rapid enough timespans to enable their therapeutic application in living organisms. The length scale of these hydrogel microenvironments makes them superior to other conventional technologies, enables optimal exchange of nutrients, waste, and secreted biomolecules to and from the cell and its surrounding environment, and enables their minimally invasive delivery via syringe injection.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

As used herein, a “composition” can include component(s) of the composition and/or reaction product(s) of two or more components of the composition.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a monomer” include aspects comprising one, two, or more monomers, unless specified to the contrary or the context clearly indicates only one monomer is included.

While the foregoing is directed to embodiments of the present disclosure, other and further embodiments of the disclosure may be devised without departing from the basic scope thereof, and the scope thereof is determined by the claims that follow.

What is claimed is:

1. A composition, comprising:
a microcapsule comprising a core and a polymeric shell enclosing the core, the core comprising a cell, and the polymeric shell comprising, in polymerized form, one or more photoreactive monomers and a linker.

2. The composition of claim 1, wherein the one or more photoreactive monomers comprise a methylene functional group, an acid functional group, or combinations thereof.

3. The composition of claim 2, wherein, when the one or more photoreactive monomers comprise a methylene functional group, the one or more photoreactive monomers comprise polyethylene glycol norbornene, polyethylene glycol diacrylate, derivatives thereof, or combinations thereof.

4. The composition of claim 2, wherein, when the one or more photoreactive monomers comprise an acid functional group, the one or more photoreactive monomers comprise polylactic acid, derivatives thereof, or combinations thereof.

5. The composition of claim 1, wherein the linker is a dithiol linker.

6. The composition of claim 5, wherein the dithiol linker is a polyethylene glycol-dithiol.

7. The composition of claim 1, wherein:
the linker has a molecular weight from about 500 Da to about 10,000 Da;
the one or more photoreactive monomers has a molecular weight from about 250 Da to about 50,000 Da; or
a combination thereof.

8. A composition, comprising:
a hydrogel comprising, in polymerized form, one or more photoreactive monomers and a thiol linker, wherein at least one of the one or more photoreactive monomers comprise a methylene functional group; and
cells dispersed within the hydrogel.

9. The composition of claim 8, wherein:
the one or more photoreactive monomers comprise polyethylene glycol norbornene, polyethylene glycol diacrylate, derivatives thereof, or a combination thereof; and
the thiol linker is a polyethylene glycol-dithiol.

10. The composition of claim 8, wherein the thiol linker has a molecular weight of about 6,000 Da or less.

11. The composition of claim 9, wherein the one or more photoreactive monomers has a molecular weight of about 30,000 Da or less.

12. The composition of claim 9, wherein the one or more photoreactive monomers comprises polylactic acid or a derivative thereof.

13. The composition of claim 8, wherein an average diameter of the hydrogel is about 500 μm or less.

14. A process for forming a composition, comprising:
introducing cells with one or more components in a microfluidic device to form a reaction mixture, the one or more components comprising a photoreactive monomer, a photoinitiator, a dithiol linker, or combinations thereof; and
polymerizing the reaction mixture by exposure to ultraviolet light, under polymerization conditions, to form a composition comprising the cells dispersed in or encapsulated within a hydrogel.

15. The process of claim 14, further comprising introducing a fluorocarbon oil with the reaction mixture.

16. The process of claim 14, wherein the photoreactive monomer comprises polyethylene glycol norbornene, polyethylene glycol diacrylate, polylactic acid, derivatives thereof, or combinations thereof.

17. The process of claim 14, wherein the polymerization conditions comprise:
a duration of exposure to the ultraviolet light that is from about 1 millisecond to about 60 seconds;
an energy density of the ultraviolet light that is from about 1 mW/cm² to about 10,000 mW/cm²; or
a combination thereof.

18. The process of claim 17, wherein the duration of exposure is less than about 30 seconds, and the energy density of the ultraviolet light is less than about 1,000 mW/cm².

19. The process of claim 14, wherein a pH of the reaction mixture is from about 5 to about 9.

20. The process of claim 14, wherein an average diameter of the hydrogel is from about 50 μm to about 200 μm.
